(19) United States
(12) Patent Application Publication
(10) Pub. No.: US 2006/0106447 A1
(43) Pub. Date: May 18, 2006

(54) Adjustable stiffness medical system

(75) Inventor: Steven W. Opolski, Carlisle, MA (US)

Correspondence Address: KIRKPATRICK & LOCKHART NICHOLSON GRAHAM LLP, STATE STREET FINANCIAL CENTER, ONE LINCOLN STREET, BOSTON, MA, 02111-2950, US

(73) Assignee: NMT Medical, Inc., Boston, MA (US)

(21) Appl. No.: 11/046,643

(22) Filed: Jan. 26, 2005

Related U.S. Application Data
(60) Provisional application No. 60/539,214, filed on Jan. 26, 2004.

(51) Int. Cl. A61F 2/06

(52) U.S. Cl. 623/1.11

(57) ABSTRACT

A delivery system for implanting a medical device within a lumen or body cavity of a patient is provided, the delivery system having a catheter with a plurality of coils, wherein the gaps between the coils can be increased or decreased, by means of an actuator, to alter the flexibility or stiffness of the catheter during delivery of the medical device.

### CROSS REFERENCE TO RELATED APPLICATIONS

This application claims priority to and benefit of U.S. provisional application 60/539,214 filed on Jan. 26, 2004, which is incorporated herein by reference in its entirety.

### FIELD OF THE INVENTION

The invention relates generally to a delivery system with adjustable stiffness, and uses therefore.

### BACKGROUND OF THE INVENTION

Modern medical technology has produced a number of medical devices that are designed for delivery into or through the vasculature using a delivery system such as a catheter. Among these medical devices are septal occluders such as those described in U.S. Pat. No. 5,425,744, the entire disclosure of which is hereby incorporated by reference. Delivery of intracardiac occluders presents special challenges for an operator. First, the occluder must be carefully and precisely deployed within the center of the defect to assure proper closure. Second, the delivery system must be capable of traversing the tortuous anatomy of the heart and vascular system, which includes small radii of curvature, for delivery of the occluder to the deployment site.

Delivery systems for medical devices such as septal occluders must therefore satisfy a number of requirements to be effective. The delivery system must have a predetermined tensile strength and stiffness in order for it to function properly. However, it must also be flexible, so that it can be guided safely to the intended target site without serious damage to surrounding tissue.

Exemplary delivery systems presently used within tortuous anatomy consist of an elongate spring type guide tube through which a single elongate core wire passes. A metal ball is formed on the distal end of the core wire. The bending stiffness of the system formed by the spring guide and the core wire is usually dominated by the relatively stiff core wire. In addition, delivery systems employing such spring-type guide tubes require the use of a safety wire to keep the spring compressed when a tensile load is applied to the delivery system. Further, the use of spring-type delivery systems generally requires the use of separate guide wire catheters to help negotiate the implant to the deployment site. This requirement necessitates the use of additional equipment, which can cause space limitations during a catheterization procedure.

A need therefore remains for a versatile catheter that has adjustable flexibility or stiffness.

### SUMMARY OF THE INVENTION

The invention provides a delivery system including a catheter that has adjustable stiffness and flexibility, such that an operator can adjust the stiffness or flexibility of the catheter as desired. The system, according to the invention, is useful for delivering medical or surgical devices including, but not limited to, intracardiac devices, e.g., septal ocluders, angioplasty balloons, intravascular stents, and artherectomy catheters, for example.

The catheter of the invention includes a plurality of coils. The catheter has a first position in which adjacent coils in a pair, such as a proximal coil and a distal coil, are spaced by a gap, and a second position in which the adjacent coils are spaced by a gap that is narrower than the gap in the first position. The catheter moves between the first position and the second position by means of an actuator on a handle. The catheter is more flexible in the first position, when the coils are spaced by the wider gap, than in the second position, when the coils are spaced by the narrower gap. In an embodiment, an elongated member is axially disposed and slidably movable in the lumen of the catheter, and an end-piece contacts a coil of the catheter. One end of the elongated member is secured to the end-piece and the other end of the elongated member is joined to the actuator. The actuator can be moved to change the force exerted on at least a portion of the catheter, thereby changing the gap space between at least some of the coils. In another embodiment, the coils are continuous, forming an integral piece, such as, for example, a helix.

The coil wire can have a cross-section of any shape. In an embodiment, the coil wire has a circular cross-section. In another embodiment, the coil wire has a rectangular cross-section. In yet another embodiment, the coil wire is made of a ribbon-like, substantially planar wire. The coils may be made of any flexible wire material, such as nitinol, stainless steel, or an alloy thereof, or may be made of one or more polymers. In an embodiment, the coils comprise a helix.

In another aspect, the invention provides a method for delivering a medical device to a patient's body lumen or tissue, for example, cardiac tissue, such as a patent foramen ovale, by (a) providing a medical device in a delivery system having a catheter comprising a plurality of coils and a lumen; the catheter being in a first position wherein at least adjacent coils, such as a proximal and a distal coil, are spaced by a first gap; (b) transitioning the catheter, by means of an actuator, from a first position to a second position wherein the at least two adjacent coils are spaced by a second gap, and the second gap is narrower than the first gap; (c) delivering the medical device to the tissue in the patient; and (d) transitioning the catheter from the second position to the first position. The delivery system including the catheter according to the invention is more rigid in the second position than it is in the first position and is more flexible in the first position than it is in the second position.

### BRIEF DESCRIPTION OF THE DRAWINGS

The foregoing and other objects, features and advantages of the present invention, as well as the invention itself, will be more fully understood from the following description of preferred embodiments when read together with the accompanying drawings, in which:

FIG. 1A depicts a longitudinal cross-section of an adjustable stiffness delivery system, according to one illustrative embodiment of the invention.

FIG. 1B depicts a longitudinal cross-section of a portion of a delivery system according to another illustrative embodiment of the invention.

FIG. 2 depicts a perspective end view of a portion of the delivery system illustrated in FIG. 1A taken along lines 2-2.

FIG. 3 depicts a longitudinal cross-section of an embodiment of an adjustable stiffness delivery system according to an illustrative embodiment of the invention, with the catheter in the second or compressed position.

FIG. 4 depicts a portion of an embodiment of a delivery system with a catheter in the second or compressed position and comprising a plurality of coils manufactured from materials having a rectangular cross-section according to an illustrative embodiment of the invention.

FIGS. 5A-5C depict an exemplary method for a percutaneous delivery of a septal occluder with the delivery system according to an illustrative embodiment of the invention.

### DETAILED DESCRIPTION

The present invention provides a delivery system for a medical device, e.g., an intracardiac septal occluder, including a catheter with adjustable stiffness. The catheter of the delivery system may be a guide catheter for delivery of, for example, angioplasty balloons, stents, or an artherectomy catheter. All of the following embodiments of the invention have a catheter including a plurality of coils that are reversibly compressible, and an actuator for manipulating the compression and relaxation of the coils thereby transitioning the catheter between a rigid and flexible state. The delivery system includes an elongated member that in one embodiment is axially moveable in the catheter lumen. The distal end of the elongated member is joined to an end-piece and the proximal end is joined to the actuator. The actuator slidably moves the elongated member relative to the catheter, thereby compressing or relaxing the coils of the catheter.

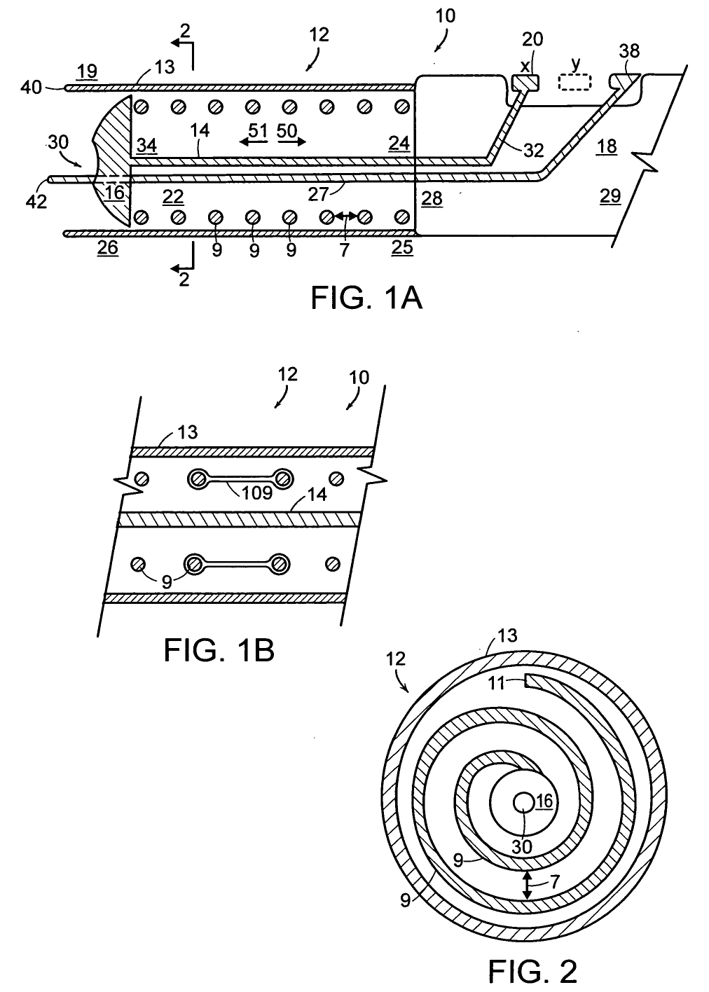

FIG. 1A depicts a longitudinal cross-section of an adjustable stiffness delivery system 10 according to an illustrative embodiment of the invention. The delivery system 10 includes a catheter 12, a handle 18, a first elongated member 14, an end-piece 16, and an actuator 20. In a particular embodiment according to the invention, the delivery system 10 includes an outer sheath 13. The catheter 12 comprises a plurality of coils 9 and a catheter lumen 22.

With continued reference to FIG. 1A, the first elongated member 14 is axially disposed and slidably movable in the catheter lumen 22. The first elongated member 14 has a proximal end 32, i.e., the end closest to the operator, operatively joined to the first actuator 20 in the handle 18, and a distal end 34 joined to the end-piece 16. The actuator 20 reversibly moves the elongated member 14 between an extended position illustrated in FIG. 1A and a retracted position (not shown) by sliding the actuator 20 on the handle 18 between position X and position Y (in ghost outline), respectively.

With continued reference to FIG. 1A, in one embodiment, the delivery system includes an outer sheath 13 and a second elongated member 27 axially movable in the catheter lumen 22. The distal end 42 of the second elongated member 27 is detachably joined to a medical device, for example, a septal occluder (not shown in FIG. 1A) and the proximal end is joined to a second actuator 38 in the handle 18, which is capable of moving the second elongated member 27 relative to the outer sheath 13, thereby deploying the medical device from the distal end 40 of the outer sheath 13. Alternatively, the second elongated member 27 is stationary and the outer sheath 13 is operatively joined to the second actuator 38 (not shown). In this alternative, the medical device is deployed from the distal end 40 of the outer sheath 13 when the second actuator 38 withdraws the moveable outer sheath 13 over the stationary secondary elongated member 27.

Referring still to FIG. 1A, the end-piece 16 may be any configuration that is joinable to the distal end 34 of the elongated member 14. For example, in one embodiment, the end-piece 16 is disc-shaped, as illustrated in FIG. 1A, or alternatively cross-shaped, or t-shaped (not shown). The end-piece 16 may fully or only partially obstruct the distal end of the catheter lumen 22 and may have a configuration, such as the opening 30 in FIG. 1A, that communicates with the lumen 22 of the catheter 12 and allows for fixation or delivery of an instrument or an implant.

FIG. 2 illustrates an end view of the catheter in FIG. 1A. In the exemplary embodiment, the coils 9 are continuous, i.e., the coils are made from a single piece of material, e.g., a single wire. In one embodiment according to the invention, the coils are turned to form a helix 11. Referring back to FIG. 1A, in one embodiment according to the invention, the coils 9 extend from the distal end 26 to the proximal end 25 of the catheter 12. Alternatively, a tube, for example, a polymer tube 109, is intermittently placed between a proximal and distal coil as shown in FIG. 1B. The region of the tube interspersed with coils 9 imparts variable regions of flexibility along the length of the catheter 12.

Referring to FIG. 1A, the coils 9 in a relaxed state are separated from adjacent coils by a first gap 7. For example, the first gap 7 may be in the range, for example, of about 0.0001 inches to about 0.1 inches in length, preferably about 0.001 inches in length. When the coils 9 are compressed, by means described below, the first gap 7 decreases in length to form a second gap that is narrower than the first gap. The second gap may be in the range, for example, of about 0 inches to about 0.001 inches, preferably about 0 inches.

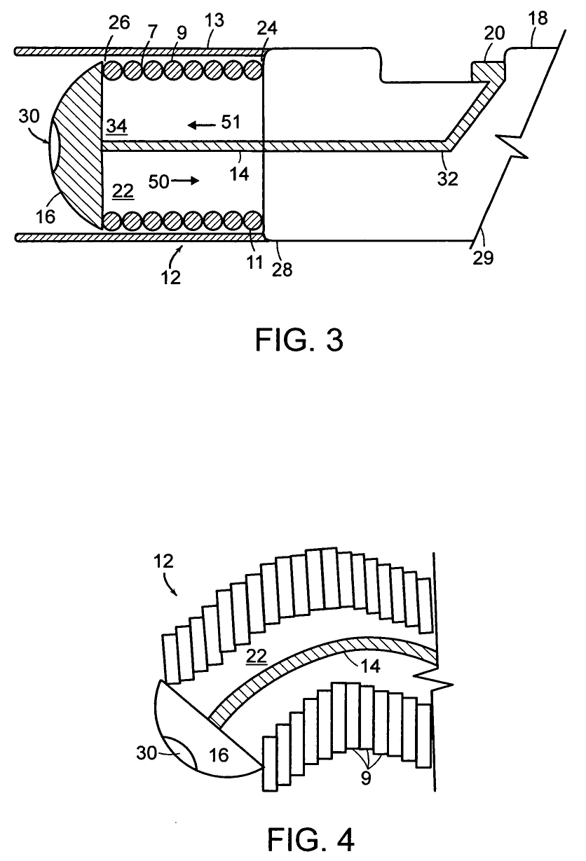

Referring now to FIG. 3, the catheter 12 can be transitioned from a relaxed state, for example as illustrated in FIG. 1A, to a compressed state, for example, as illustrated in FIG. 3. The catheter 12 transitions from the relaxed state to the compressed state when the operator moves the first elongated member 14 in the direction indicated by the arrow 50, for example, by manually moving the first actuator 20 on the handle 18 toward the proximal end 29 of the handle 18. The gap 7 between the coils 9 is thereby reduced in length in the compressed state illustrated in FIG. 3 compared to the gap 7 between the coils 9 in the relaxed state of the catheter 12 illustrated in FIG. 1A. In an embodiment, the gap 7 is substantially the same from coil 9 to adjacent coil 9. Alternatively, the gap 7 between sets of two adjacent coils may be different.

Referring to FIG. 1A and FIG. 3, the axial stiffness of the catheter 12 according to the invention is adjusted by altering the magnitude of force applied to the first elongated member 14. The greater the magnitude of force directed in the proximal direction by the first actuator 20, to the first elongated member 14, and to the end-piece 16, the shorter the gap 7 between the coils 9, which increases the stiffness or rigidity of the catheter 12. Conversely, the greater the magnitude of force directed to the end-piece 16 through the first elongated member 14 by the first actuator 20 in the distal direction, the larger the gap 7 between the coils 9, which increases the flexibility of the catheter 12. Increasing catheter stiffness requires that the coils be in close proximity. Accordingly, when the gap between the coils 9 is reduced to nothing, i.e., the coils 9 are touching, the compressive (e.g., normal) force between the coils creates a friction force (e.g., lateral friction force) and provides for an increase in stiffness (e.g., when the gap is essentially zero and compressive force is greater than zero), with higher force linked to greater catheter stiffness.

The coils 9 of the invention may be manufactured with any metallic material, such as nickel-titanium (nitinol), stainless steel, vanadium, iron, gold, platinum, tantalum, tungsten, iridium, cobalt, molybdenum, chromium, or an alloy thereof. In addition, the coils may be made of a superelastic or pseudoelastic copper alloy, such as Cu—Al—Ni, Cu—Al—Zi, and Cu—Zi, for example. In an embodiment, the coils 9 of the invention comprise at least one polymer, such as, for example, polyimide, polyethylene, polyurethane, tetrafluoroethylene (TFE), polytetrafluoroethylene (PTFE), expanded polytetrafluoroethylene (ePTFE), or a mixture or a coating thereof. The coils may be continuous, as in a helix, or adjacent coils, e.g., adjacent rings, which may be interconnected by means of a material such as ePTFE.

The length and width of a coil 9 depends upon its intended use. The coil 9 may be between about 0.25 inches and about 150 inches in length, for example. The width of the material of a coil 9, e.g., a wire, may have a transverse diameter of between about 0.001 inches to about 0.040 inches, for example, and may have any cross-sectional shape, for example, circular, semi-circular, square, rectangular, oval, semi-oval, triangular, polygonal, or substantially planar (i.e., ribbon-like).

Referring to the embodiment illustrated in FIG. 4, materials of coils 9, such as a wire, that have a substantially planar or rectangular cross-section and have flat surfaces provide a higher friction force between coils 9 in the compressed state than wire coils 9 that have a round cross-section. Thus, in an embodiment, rectangular or substantially planar coils 9 provide increased stability because a flat surface provides greater points of contact and therefore greater friction between the flat surface and another surface than a round surface.

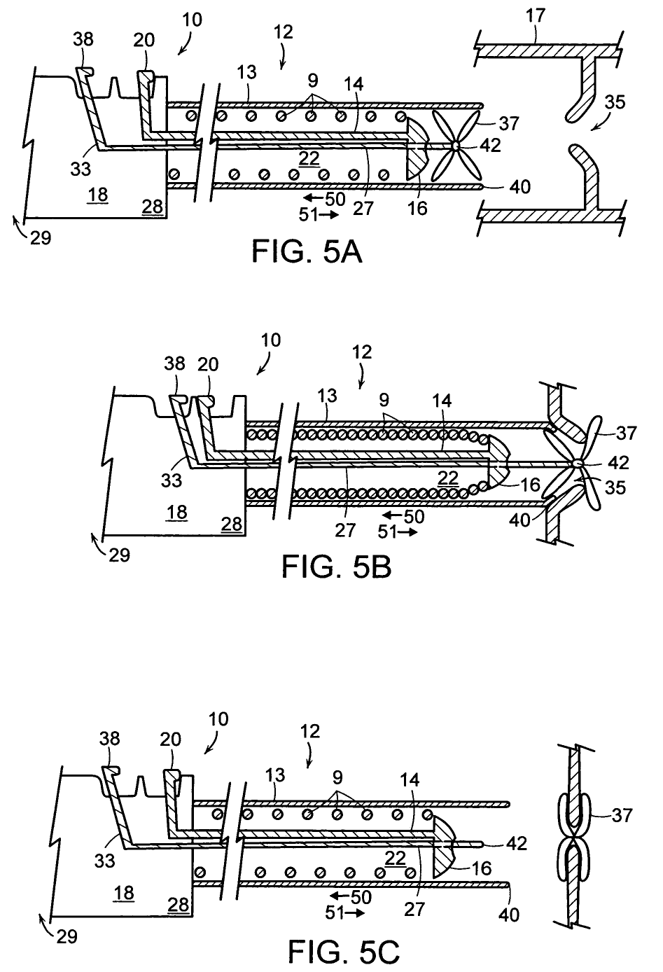

FIGS. 5A-5C depict an exemplary method for the percutaneous delivery of a medical implant, e.g., a septal occluder 37, by the delivery system 10 according to the invention. The method may be used for delivering or retrieving a medical device or implant, such as a septal occluder 37, to an anatomical site in the body of a patient, for example, a patent foramen ovale in the heart of a patient. The embodiment of the delivery system 10 depicted in FIG. 5A includes a handle 18, a catheter 12, an outer sheath 13, a first actuator 20 joined to a first elongated member 14, a second actuator 38 joined to a second elongated member 27, and an end-piece 16. The catheter 12 includes a plurality of coils 9 and a catheter lumen 22.

Referring now to FIGS. 5A and 5B, when increased rigidity is required, for example, when the distal end 26 of the catheter 12 encounters a valve or hole 35, the operator applies force to the first elongated member 14 in the direction 50, toward the proximal end 29 of the handle 18. The proximal-directed force moves the coils 9 closer together, for example, such that the gap 7 between the coils 9 is eliminated and the coils 9 are touching. In this coil configuration, the catheter 12 is in a second or compressed position and is less flexible (i.e., more rigid) and can more easily penetrate a valve or hole 35. As the magnitude of proximal-directed force on the first elongated member 14 is increased, the catheter 12 stiffness increases.

Referring now to FIG. 5B, when the anatomical deployment site is reached, for example a patent foramen ovale, the second elongated member 27 and detachably joined medical device, for example a septal occluder 37, are extended beyond the distal end 40 of the outer sheath 13 by moving the second actuator 38 toward the handle 18 distal end 28. The septal occluder 37, which is detachably joined to the distal end 42 of the second elongated member 27, is thereby deployed at the anatomical deployment site.

Referring now to FIG. 5C, once the septal occluder 37 has been inserted at the deployment site, greater flexibility of the catheter 12 is required for its removal. The catheter 12 is transitioned from the compressed position to the relaxed position to obtain greater flexibility during removal of the delivery device. In order to increase catheter 12 flexibility, the first elongated member 14 is moved distally causing the coils 9 to move apart, returning the catheter 12 to the first or relaxed position.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are embraced therein.

### INCORPORATION BY REFERENCE

All publications and patent documents cited in this application are incorporated by reference in their entirety for all purposes to the same extent as if the content of each individual publication or patent document was incorporated herein.

What is claimed is:

1. A system for delivering a medical device, comprising:
a handle;
a catheter comprising a plurality of coils, and a lumen;
the catheter further comprising a first position wherein at least one pair of adjacent coils are spaced by a first gap, and a second position wherein the adjacent coils are spaced by a second gap narrower than the first gap; and
an actuator positioned on the handle for moving the catheter between the first position and the second position, wherein the catheter is more flexible in the first position than in the second position.

2. The system as in claim 1, wherein the catheter further comprises an end-piece that contacts one of the plurality of coils, and an elongated member axially disposed and slidably movable in the lumen of the catheter, one end of the elongated member being secured to the end-piece, and the other end of the elongated member being joined to the actuator.

3. The system as in claim 1, wherein the coils are continuous.

4. The system as in claim 1, where the plurality of coils comprise a plurality of rings.

5. The system as in claim 1, wherein the coils comprise a wire with a circular cross-section.

6. The system as in claim 1, wherein the coils comprise a wire with a rectangular cross-section.

7. The system as in claim 1, wherein the coils form a helix.

8. The system as in claim 1, wherein the coils comprise a ribbon-like wire.

9. The system as in claim 1, wherein the coils comprise nitinol.

10. The system as in claim 1, wherein the coils comprise stainless steel.

11. The system as in claim 1, further comprising an outer sheath.

12. The system as in claim 1, further comprising a second elongated member.

13. The system as in claim 1, wherein the coils comprise at least one polymer.

14. The system as in claim 1, wherein the first gap is from about 0.0001 inches to about 0.1 inches in length and the second gap is about 0 inches.

15. A method for delivering a medical device to a tissue in a patient, the method comprising the steps of:
a. providing a medical device in a delivery system, the delivery system comprising:
a catheter comprising a plurality of coils, and a lumen, the catheter being in a first position wherein at least two adjacent coils are spaced by a first gap;
b. transitioning the catheter by means of an actuator from the first position to a second position wherein the at least two adjacent coils are spaced by a second gap, and the second gap is narrower than the first gap;
c. delivering the medical device to the tissue in the patient; and
d. transitioning the catheter from the second position to the first position.

16. The method of claim 15, wherein the catheter further comprises an end-piece that contacts one of the plurality of coils, and an elongated member axially disposed and slidably movable in the lumen of the catheter, one end of the elongated member being secured to the end-piece, the other end of the elongated member being joined to the actuator.

17. The method as in claim 15, wherein the method is used to deliver a septal occluder to a heart.
